(19) United States
(12) Patent Application Publication
(10) Pub. No.: US 2024/0316542 A1
(43) Pub. Date: Sep. 26, 2024

(54) Zn-METAL OXIDE/ZEOLITE CATALYST AND METHODS OF MAKING THEREOF

(71) Applicant: The Regents of the University of California, Oakland, CA (US)

(72) Inventors: Ji Yang, Berkeley, CA (US); Ji Su, Richmond, CA (US)

(21) Appl. No.: 18/603,475

(22) Filed: Mar. 13, 2024

Related U.S. Application Data
(60) Provisional application No. 63/491,232, filed on Mar. 20, 2023.

(51) Int. Cl. B01J 29/78; B01J 23/26; B01J 37/00; B01J 37/04; B01J 37/08; C01B 32/40; C01B 39/02; C07C 5/42

(52) U.S. Cl. CPC B01J 29/783; B01J 23/26; B01J 37/0036; B01J 37/04; B01J 37/08; C01B 32/40; C01B 39/026; C07C 5/42; B01J 2229/38; C07C 2529/78

(57) ABSTRACT

This disclosure provides systems, methods, and apparatus related to catalysts. In one aspect, a catalyst is a ZnxMy/zeolite catalyst, with ½≤x/y≤4, and M being a metal from a group Cr, Mo, and W. Zn is an oxide, M is an oxide, and there are at least some ZnMOₓ oxides.

### RELATED APPLICATIONS

This application claims priority to U.S. Provisional Patent Application No. 63/491,232, filed Mar. 20, 2023, which is herein incorporated by reference.

### STATEMENT OF GOVERNMENT SUPPORT

This invention was made with government support under Contract No. DE-AC02-05CH11231 awarded by the U.S. Department of Energy. The government has certain rights in this invention.

### BACKGROUND

The synergistic effects generated by mixed or supported bifunctional catalysts are often claimed to promote the catalytic performance of traditional heterogeneous catalysts. Recently, the construction of synergistic pair-sites with colocalized metal atoms to facilitate distinct elementary steps in the catalytic reaction has been established as a step toward atomically synergistic bifunctional catalyst development. The interaction between these adjacent metal atoms, similar to the metal-support interaction of traditional heterogeneous catalysts, offers the possibility to modulate their respective electronic structure to further enhance their catalytic activity. However, precisely controlling their colocalization requires a complicated synthesis process with multiple synthesis steps to load single atoms. Moreover, the stability of the well-defined synergistic sites is debatable, especially under harsh reaction conditions such as high reaction temperatures.

Growing CO₂ emissions and abundant shale gas reserves have prompted a significant amount of research to explore efficient approaches for co-utilizing the products to produce value-added chemicals. Ethane, the second-largest component of shale gas, is an ideal alternative hydrogen source for CO₂ conversion. The co-conversion of ethane and CO₂ (C₂H₆+CO₂→C₂H₄+CO+H₂O) is a viable alternative to the ethane steam cracking for ethylene production under the goal of net negative CO₂ emissions. Further, iso-stoichiometric co-conversion of ethane and CO₂ (ICEC) to ethylene and CO is crucial for direct downstream processes such as the hydroformylation reaction to produce aldehyde and polymerization process to produce polyketones.

However, the co-conversion process lacks a viable catalyst for achieving high ethylene selectivity and CO₂ utilization simultaneously. Metal-based catalysts suffer from the inevitable cleavage of C—C bonds through dry reforming pathways and lower the ethylene selectivity. Oxide-based catalysts exhibit the great merit of preferential C—H bond scission over C—C bond scission pathways but require excessive CO₂ cofeeding to reduce ethane adsorption and scavenge the surface H species.

Specifically, Zn and Cr oxide-based catalysts were widely studied for co-conversion of ethane and CO₂. The acidic Zn²⁺ site displayed high activity for C—H bond cleavage in ethane and CO₂ activation, requiring the participation of adjacent active sites to form binuclear sites. The challenge is that acidified Zn²⁺—H hydride displays a capacity for C—C bond scission of ethane, leading to undesired production of methane. Redox Cr⁶⁺ sites require lattice oxygen as a H acceptor to dissociate C—H bonds but trigger the formation of less active Cr³⁺ species. The reoxidation of Cr³⁺ to Cr⁶⁺ is limited by slow O abstraction from CO₂.

### SUMMARY

The above analyses indicate the need for an atomically-synergistic binuclear a Zn—O— metal (e.g., Zn—O—Cr) site catalyst for ICEC, with Zn facilitating CO₂ adsorption and activation to provide O species for Cr⁶⁺ regeneration and adjacent Cr as an electron donor reducing the acidity of Zn²⁺ to facilitate its activity and selectivity for C—H bond scission. More generally, exploring a cooperative redox and acid-base catalytic mechanism for ICEC is highly desirable.

Described herein are the fabrication and demonstration of a Zn—O-metal (e.g., Zn—O—Cr) atomically-synergistic binuclear-site catalyst (ABC) that is efficient for ICEC with high ethylene selectivity and utilization of converted CO₂ (U_{CO}_{₂}). Compared with pure Zn and Cr catalysts, Zn—O—Cr ABC displays ˜1.5 and ˜4-fold higher catalytic activity with 100% ethylene selectivity and 99.0% U_{CO}_{₂}, under optimized reaction conditions. A factor in this high performance is the discovery that Cr facilitates the formation of a Zn^{δ+}(0<δ<2) site to enhance the β-C—H bond cleavage of ethane, while the resulting Lewis base Zn—H^{δ-} hydride favors CO₂ adsorption and activation, and prevents C—C bond scission of ethane. The redox Cr site accelerates CO₂ dissociation and facilitates H₂O formation/desorption. The apparent activation energies of ethane conversion and CO₂ conversion are ˜70.9 and ˜74.0 KJ/mol, which demonstrates the rate matching achieved in ICEC.

One innovative aspect of the subject matter described in this disclosure is a ZnxMy/zeolite catalyst, with ½≤x/y≤4, with M being a metal from a group Cr, Mo, and W, with Zn being an oxide, with M being an oxide, and with at least some ZnMOₓ oxides.

Another innovative aspect of the subject matter described in this disclosure can be implemented in a method including mixing a Zn precursor, a metal precursor, and a zeolite to form a mixture. A metal of the metal precursor is a metal from a group Cr, Mo, and W. The mixture is then ground. After grinding the mixture, the mixture is heat treated to form a ZnxMy/zeolite catalyst, with ½≤x/y≤4. Zn is an oxide, M is an oxide, and Zn and M form at least some ZnMOₓ oxides.

Another innovative aspect of the subject matter described in this disclosure can be implemented in a method including providing a ZnxMy/zeolite catalyst, with ½≤x/y≤4. M is a metal from a group Cr, Mo, and W. Zn is an oxide, M is an oxide, and there are at least some ZnMOₓ oxides. The ZnxMy/zeolite catalyst is heated to about 400° C. to 650° C. Carbon dioxide and ethane are flowed through the ZnxMy/zeolite catalyst to generate carbon monoxide and ethylene.

Details of one or more embodiments of the subject matter described in this specification are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

FIG. 1A shows a representative TEM image, FIG. 1B shows a representative HAADF-STEM image, and FIGS. 1C and 1D show EDS mapping of Zn—O—Cr ABC with Zn/Cr ratio of 3/1. FIG. 1E shows the proportion of Zn^{δ+}(0<δ<2) (left) and Cr⁶⁺ (right) with varying Zn/Cr ratios (Results derived from Auger spectra of Zn LMM and Cr 2p_{3/2} XPS spectra). FIGS. 1F and 1G show enlarged electron transition features in Zn and Cr K-edge XANES spectra. FIG. 1H shows k³-weighted Fourier-transformed extended X-ray absorption fine structure (FT-EXAFS) spectra (Zn K-edge) of Zn—O—Cr ABCs with varying Zn/Cr ratios, with Zn foil and ZnO as references. FIG. 1I shows the coordination number (CN) of Zn—O—Cr(Zn) and Cr—O in Cr⁶⁺state in Zn—O—Cr ABCs with varying Zn/Cr ratios.

FIG. 2A shows a table that includes the catalytic performance of Zn—O—Cr ABCs (Reaction conditions: Temperature=550° C.; Reactant composition=5% C₂H₆+5% CO₂+90% Ar; WHSV=7500 mL g_{cat}⁻¹ h⁻¹). FIG. 2B shows C₂H₆ and CO₂ conversion, utilization of converted CO₂ (U_{CO}_{₂}) of Cr/SSZ-13, Zn/SSZ-13, and Zn₃Cr₁/SSZ-13 catalysts in ICEC at 550° C. FIG. 2C shows the reaction temperature dependent performance of Zn₃Cr₁/SSZ-13 catalyst. FIG. 2D shows Arrhenius plots of Zn₃Cr₁/SSZ-13 catalyst (obtained when both C₂H₆ and CO₂ conversions are <10%). FIG. 2E shows C₂H₆ conversion and C₂H₄ selectivity over Zn₃Cr₁/SSZ-13, Zn/SSZ-13, and Cr/SSZ-13 with (black columns) and without (gray columns) CO₂ at 550° C.

FIGS. 3A-3C show in situ ambient pressure X-ray photoelectron spectroscopy (APXPS): FIG. 3A, C1s spectra; FIG. 3B, Auger spectra of Zn LMM; and FIG. 3C, Cr 2p3/2 spectra as a function of reaction conditions for Zn₃Cr₁/SSZ-13. T=550° C.: ultra-high vacuum (UHV), C₂H₆ (50 mTorr)+CO₂ (50 mTorr), C₂H₆ (100 mTorr), and CO₂ (100 mTorr) in sequence. FIG. 3D shows a schematic illustration of electron transfer from Zn to H after the 2^{nd} C—H bond scission in ethane, where electron accumulation (Δρ=+1×10⁻³ e bohr⁻³) and depletion (Δρ=−1×10⁻³ e bohr⁻³) and (Δρ=−1×10⁻³ e bohr⁻³) being represented. FIG. 3E shows a schematic illustration of the oxidation of Cr in Zn—O—Cr when decomposing COOH* intermediate.

FIGS. 4A and 4B show examples of illustrations of the atomically synergistic mechanism of ICEC over binuclear Zn—O—Cr sites. FIG. 4A shows an illustration of the catalytic cycles of ICEC over binuclear Zn—O—Cr sites of Zn₃Cr₁/SSZ-13. FIG. 4B shows an illustration of the calculated energy profiles of ICEC on Zn₃Cr₁/SSZ-13, Zn/SSZ-13, and Cr/SSZ-13.

FIG. 5 shows an example of a flow diagram illustrating a manufacturing process for an Zn-metal oxide/zeolite catalyst.

FIG. 6 shows an example of a schematic illustration of a manufacturing process from a Zn—Cr oxide/SSZ-13 zeolite catalyst.

FIG. 7 shows an example of a flow diagram illustrating a method of co-conversion of ethane to CO₂ using a Zn-metal oxide/zeolite catalyst.

### DETAILED DESCRIPTION

Reference will now be made in detail to some specific examples of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise.

The terms “about” or “approximate” and the like are synonymous and are used to indicate that the value modified by the term has an understood range associated with it, where the range can be ±20%, ±15%, ±10%, ±5%, or ±1%. The terms “substantially” and the like are used to indicate that a value is close to a targeted value, where close can mean, for example, the value is within 80% of the targeted value, within 85% of the targeted value, within 90% of the targeted value, within 95% of the targeted value, or within 99% of the targeted value.

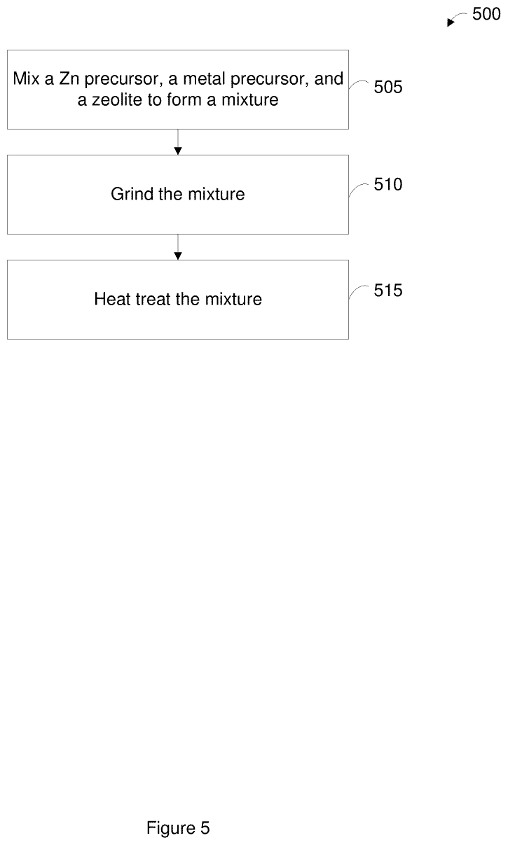

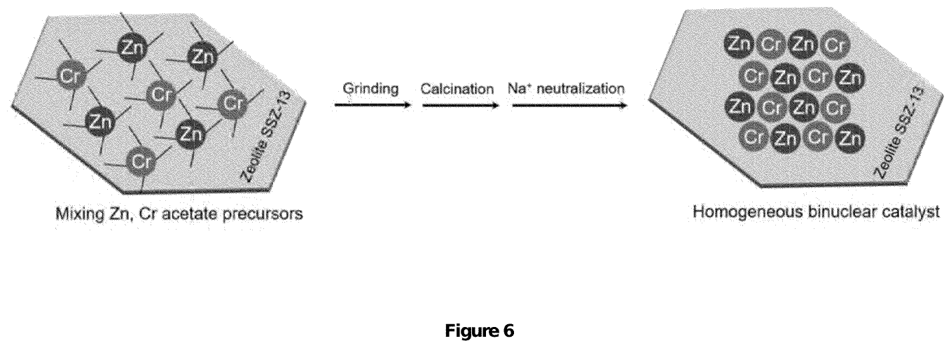

FIG. 5 shows an example of a flow diagram illustrating a manufacturing process for an Zn-metal oxide/zeolite catalyst. FIG. 6 shows an example of a schematic illustration of a manufacturing process from a Zn—Cr/SSZ-13 zeolite catalyst. Starting at block 505 of the process 500 shown in FIG. 5, a Zn precursor, a metal precursor, and a zeolite are mixed to form a mixture. A metal of the metal precursor is a metal from a group Cr, Mo, and W.

In some embodiments, the Zn precursor is a Zn precursor from a group zinc (II) acetate, zinc (II) nitrate, and zinc (II) chloride. In some embodiments, the Cr precursor is a Cr precursor from a group chromium (III) acetate hydroxide, chromium (III) nitrate, and chromium (III) chloride. In some embodiments, the Mo precursor comprises or essentially consists of ammonium molybdate tetrahydrate. In some embodiments, the W precursor comprises or essentially consists of ammonium metatungstate hydrate.

In some embodiments, the salt of the Zn precursor and the salt of the metal precursor are the same salts. For example, zinc (II) nitrate and chromium (III) nitrate. In some embodiments, the salt of the Zn precursor and the salt of the metal precursor are different salts. For example, zinc (II) nitrate and chromium (III) chloride.

In some embodiments, the zeolite is a zeolite from a group HSSZ-13 zeolite, NaSSZ-13 zeolite, HZSM-5 zeolite, NaZSM-5 zeolite, H-ZSM-11 zeolite, H-ZSM-22 zeolite, H-ZSM-23 zeolite, ZSM-35 zeolite, Beta zeolite, SAPO-11 zeolite, and SAPO-34 zeolite. In some embodiments, the zeolite comprises a SSZ-13 zeolite.

At block 510, the mixture is ground. In some embodiments, the grinding is performed with a ball mill. In some embodiments, the grinding is performed with a mortar and pestle.

In some embodiments, the Zn precursor and the metal precursor each have a particle size of less than about 0.5 nanometers after the grinding. In some embodiments, the zeolite has a particle size of about 1 micron to 2 microns after the grinding.

At block 515, after grinding the mixture, the mixture is heat treated to form a ZnxMy/zeolite catalyst, with ½≤x/y≤4, with Zn being an oxide, with M being an oxide, and with Zn and M forming at least some ZnMOₓ oxides. In some embodiments, the heat treatment comprises heating the mixture to about 300° C. to 800° C., or about 550° C., for about 1.5 hours to 4.5 hours, or about 3 hours. In some embodiments, the heat treatment is performed in an inert atmosphere. In some embodiments, the heat treatment is performed under flowing nitrogen.

In some embodiments, the ZnMOₓ oxides are solid solution oxides. That is, the Zn, M, and oxygen form a solid solution. In some embodiments, the ZnMOₓ oxides have a structure of Zn^{δ+}—O-M⁶⁺ (0<δ<2). In some embodiments, the catalyst is a Zn₃M₁/zeolite catalyst. In some embodiments, the catalyst is a Zn₃Cr₁/SSZ-13 catalyst.

In some embodiments, only ZnMOₓ oxides are formed, and no Zn oxides or metal oxides are formed. In some embodiments, only dispersed ZnMOₓ oxides with a structure of Zn^{δ+}—O-M⁶⁺ (0<δ<2) are formed, and no Zn oxides or metal oxides are formed. This is the case, for example with Zn₃Cr₁/zeolite catalysts, including a Zn₃Cr₁/SSZ-13 catalyst.

In some embodiments, the zeolite catalyst comprises a zeolite with Zn oxide nanoparticles, M oxide nanoparticles, and ZnMOₓ oxide nanoparticles disposed on the zeolite. In some embodiments, the Zn oxide nanoparticles, the M oxide nanoparticles, and the ZnMOₓ oxide nanoparticles have particle sizes of about 0.5 nanometers to 1 nanometer.

In some embodiments, the zeolite catalyst comprises a zeolite with ZnMOₓ oxide nanoparticles disposed on the zeolite. In some embodiments, the ZnMOₓ oxide nanoparticles have particle sizes of about 0.5 nanometers to 1 nanometer.

In some embodiments, the surface acidity of the zeolite is neutralized. A process for neutralizing the surface acidity of the zeolite is described further below. In some embodiments, after neutralizing the surface acidity of the zeolite, surfaces of the zeolite have Na⁺ ions attached thereto.

In some embodiments, the process 500 further comprises dehydrating the zeolite prior to block 500. In some embodiments, the dehydrating comprises heating the zeolite to about 60° C. to 180° C., or about 120° C., for about 1.5 hours to 4.5 hours, or about 3 hours, while under a vacuum.

In some embodiments, the process 500 further comprises neutralizing the surface acidity of the zeolite after block 515. For example, some zeolites that are used in the embodiments described herein are proton-type zeolites that have a high surface acidity. Proton-type zeolites include HSSZ-13 zeolite, HZSM-5 zeolite, H-ZSM-11 zeolite, H-ZSM-22 zeolite, H-ZSM-23 zeolite, and Beta zeolite. In some embodiments, neutralizing the surface acidity of the zeolite comprises Na⁺-neutralizing the zeolite. In some embodiments, Na⁺-neutralizing the zeolite comprising dispersing the mixture in deionized water to form a suspension, adding a sodium bicarbonate solution to the suspension so that the suspension has a pH of about 7, and removing the mixture from the suspension.

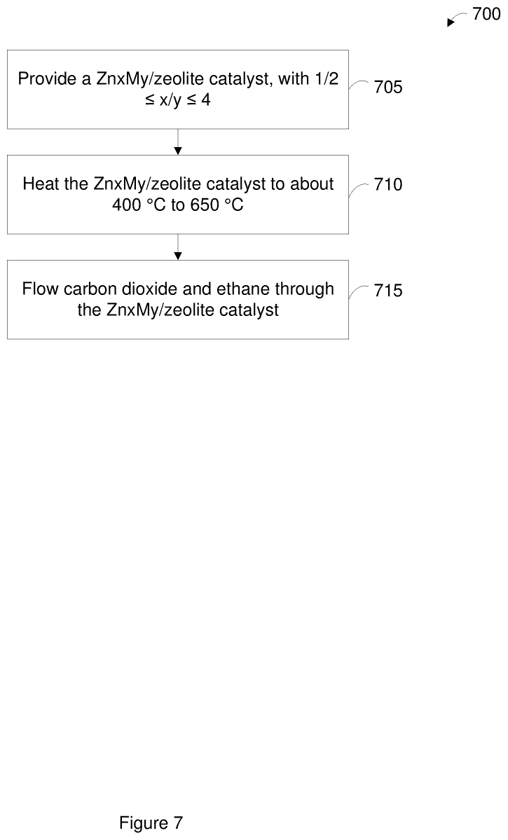

FIG. 7 shows an example of a flow diagram illustrating a method of co-conversion of ethane to CO₂ using a Zn-metal oxide/zeolite catalyst. Starting at block 705 of the process 700 shown in FIG. 7, a ZnxMy/zeolite catalyst is provided, with ½≤x/y≤4, with M being a metal from a group Cr, Mo, and W, with Zn being an oxide, with M being an oxide, and with at least some ZnMOₓ oxides.

The process 700 may be performed with any of the zeolite catalysts described herein. For example, in some embodiments, the zeolite is a zeolite from a group HSSZ-13 zeolite, NaSSZ-13 zeolite, HZSM-5 zeolite, NaZSM-5 zeolite, H-ZSM-11 zeolite, H-ZSM-22 zeolite, H-ZSM-23 zeolite, ZSM-35 zeolite, Beta zeolite, SAPO-11 zeolite, and SAPO-34 zeolite. In some embodiments, the zeolite catalyst is a Zn₃M₁/zeolite catalyst. In some embodiments, the zeolite catalyst is a Zn₃Cr₁/SSZ-13 catalyst.

At block 710, the ZnxMy/zeolite catalyst is heated to about 400° C. to 650° C. In some embodiments, the zeolite catalyst is heated in a furnace.

At block 715, carbon dioxide and ethane are flowed through the ZnxMy/zeolite catalyst, generating carbon monoxide and ethylene. In some embodiments, the ratio of carbon dioxide to ethylene is about 5 to 1 to about 1 to 1.

The following examples are intended to be examples of the embodiments disclosed herein, and are not intended to be limiting.

### Example—Materials Synthesis

ZnₓCr_{y}/SSZ-13 (x=1, y=0; x/y=½, 1, 2, 3, and 4; x=0, y=1) catalysts were prepared by a dry-deposition method. The total amount of Zn and Cr in all catalysts was set at 0.8 mmol per 1 gram (g) of support materials.

In a typical synthesis, the SSZ-13 zeolite support was pretreated in a vacuum at 120° C. for 3 h to remove moisture. A stoichiometric amount of Zinc (II) acetate and/or chromium (III) acetate hydroxide (Zn+Cr=0.8 mmol) was mixed with 1 g of SSZ-13 zeolites using an analog vortex mixer. The resulting solid mixtures were ground in glove box for 20 minutes. Subsequently, the samples were heated in flowing N₂ (100 mL/min) at 550° C. for 3 hours at a ramping rate of 2° C./min and subsequently in flowing air (100 ml/min) for another 3 hours. Both N₂ and air were purified by moisture trap. Afterward, the resulting materials were pretreated through Na⁺-neutralization. Typically, the sample was firstly dispersed in deionized water and then dropwise addition of sodium bicarbonate solution (0.01 M, pH=8) was performed until the solution reached a pH of 7. The slurry was then collected and dried at 110° C. overnight. The samples were finally calcined in static air at 500° C. for 2 hours with a ramping rate of 2° C./min. The final products were stored in an N₂ box. The control samples of ZnₓCr/SSZ-13 (x=½, 1, and 3) were synthesized using a co-precipitation (CP) method, followed by Na⁺ neutralization.

### Example—Catalytic Performance Experiments

Catalytic performance experiments for the iso-stoichiometric co-conversion of ethane and CO₂ (ICEC) were conducted in a continuous fix-bed reactor in the temperature range of 400° C. to 550° C. and under ambient pressure using an electric furnace with temperature controlled by a K-type thermocouple. In a typical catalytic measurement, a total of 200 mg catalyst diluted with 800 mg sand was loaded into the middle of the reactor plugged by quartz wool on two sides. Before the catalytic test, the catalyst bed was pretreated under a flow of Argon (25 sccm) at 550° C. for 1 hour with a ramping rate of 10° C. from room temperature. Afterward, the reactant mixtures including 5% CO₂ and 5% C₂H₆ balanced with argon were introduced at a total flow rate of 25 sccm. Argon was used as an internal standard. The reaction products were analyzed with a gas chromatograph equipped with a thermal conductivity detector (TCD) and a flame ionization detector (FID).

To better reveal the reaction kinetics, apparent activation barriers were determined in the temperature range of 400° C. to 475° C. (the conversions of CO₂ and C₂H₆ are less than 10%). The carbon and oxygen balances were within 100±2% for all tests. The conversions of CO₂ and C₂H₆, C₂H₄ selectivity, utilization of converted CO₂ (U_{CO}_{₂}), C₂H₄ yield, the turnover frequency (TOF) of C₂H₄ formation, and the space time yield (STY) of C₂H₄ formation were calculated as follows (where n is the molar flow of substance (mol/min), n_{Zn+Cr} is the total molar loading of Zn and/or Cr, M_{C}_{₂}_{H}_{₄} is the molecular weight of C₂H₄ (28 g/mol), and m_{cat} is the catalyst mass (kg)):

$${CO}_{2} conversion ( \% ) = \frac{{n}_{{CO}_{2} input} - {n}_{{CO}_{2} output}}{{n}_{{CO}_{2} input}} \times 100 \% \qquad ( 1 )$$

$${C}_{2} {H}_{6} conversion ( \% ) = \frac{{n}_{{C}_{2} {H}_{6} input} - {n}_{{C}_{2} {H}_{6} output}}{{n}_{{C}_{2} {H}_{6} input}} \times 100 \% \qquad ( 2 )$$

$${C}_{2} {H}_{4} selectivity ( \% ) = \frac{{n}_{{C}_{2} {H}_{4} output}}{{n}_{{C}_{2} {H}_{6} input} - {n}_{{C}_{2} {H}_{6} output}} \times 100 \% \qquad ( 3 )$$

$${U}_{{CO}_{2}} ( \% ) = \frac{{CO}_{2} conversion}{{C}_{2} {H}_{6} conversion} \times 100 \% \qquad ( 4 )$$

$${C}_{2} {H}_{4} yield ( \% ) = {C}_{2} {H}_{6} conversion \times {C}_{2} {H}_{4} selectivity \qquad ( 5 )$$

$$TOF = \frac{{n}_{{C}_{2} {H}_{4} output} \times 60}{{n}_{Zn + Cr}} \qquad ( 6 )$$

$$STY = \frac{{n}_{{C}_{2} {H}_{4} output} \times {M}_{{C}_{2} {H}_{4}} \times 60}{1000 \times {m}_{cat}} \qquad ( 7 )$$

It is noted that calculations of U_{CO}_{₂} were also calibrated and examined by the equation:

$${U}_{{CO}_{2}} ( \% ) = \frac{{n}_{CO output}}{{n}_{{C}_{2} {H}_{6} input} \cdot {n}_{{C}_{2} {H}_{6} output}} \times 100 \% \qquad ( 8 )$$

### Example—Controlling Zn and Cr Coordination Structure

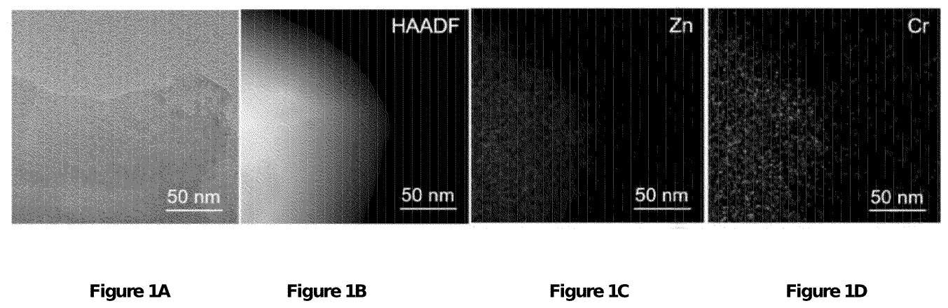

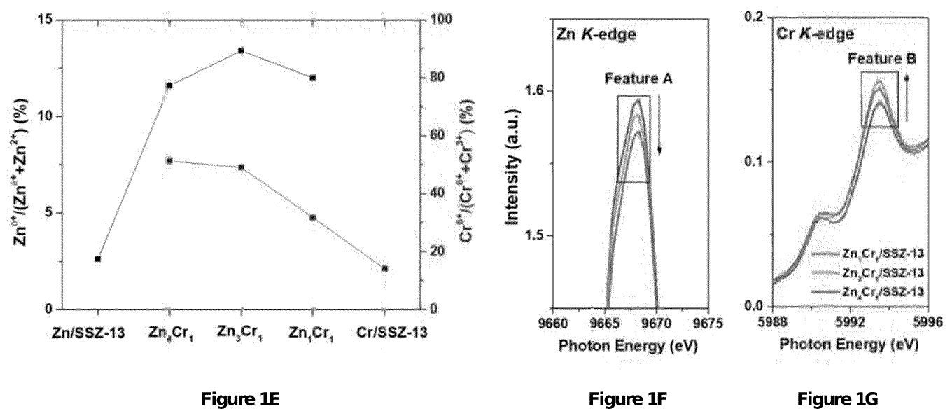

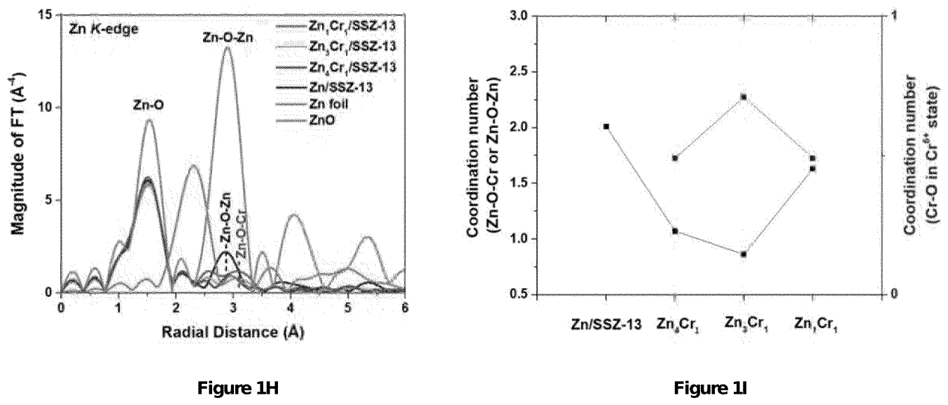

The catalysts described in the Examples are denoted as ZnₓCr_{y}/SSZ-13, where x/y refers to the ratio of Zn/Cr (x=1, y=0; x/y=½, 1, 2, 3, 4; x=0, y=1). A transmission electron microscopy (TEM) image (FIG. 1A), a scanning TEM (STEM) image, and energy dispersive spectroscopy (EDS) elemental mappings (FIGS. 1C and 1D) show high dispersion of Zn and/or Cr oxide phases, with no observable sintering of oxide nanoparticles.

X-ray diffraction (XRD) results showed the absence of spinel ZnCr₂O₄ and zincite ZnO for all dry-deposition synthesized catalysts. Cr₂O₃ phases with R3c space group were only observed in Cr/SSZ-13 and Zn₁Cr₂/SSZ-13. The control catalysts with same amount Zn and Cr precursors were prepared via a co-precipitation method (CP), followed by Na⁺ neutralization. ZnCrOₓ nanoparticles are observed in CP-synthesized catalysts. Aa phase transition from spinel ZnCr₂O₄ to ZnO was seen with CP-synthesized catalysts with Zn/Cr ratios varying from 1/2 to 3/1.

The formation of Zn^{δ+} (0<δ<2) was confirmed through Auger spectra of Zn LMM. Subpeaks at 987.5 and 990.0 eV in Auger spectra of Zn LMM were assigned to the Zn²⁺ and Zn^{δ+} (0<δ<2), respectively; the subpeaks at ˜576 and ˜580 eV in Cr 2p_{3/2} XPS were assigned to the Cr³⁺ and Cr⁶⁺, respectively. FIG. 1E shows the proportion of Zn^{δ+} (0<δ<2) and Cr⁶⁺ relative to the total amount of (Zn^{δ+}+Zn²⁺) and (Cr⁶⁺+Cr³⁺) in ZnₓCr_{y}/SSZ-13 catalysts, respectively. These proportions vary with Zn/Cr ratios, with the highest Zn^{δ+} (0<δ<2) proportion generated at Zn/Cr ratio of 3/1 and the highest Cr⁶⁺ proportions produced at Zn/Cr ratios of 3/1 and 4/1. It is hypothesized that the proximal electronic interactions between Zn and Cr could modify their respective oxidation states. This is consistent with the Zn L₃-edge X-ray absorption near-edge structure (XANES) spectra analyses, which suggests a decrease in the oxidation state of Zn in Zn Cr/SSZ-13 catalysts compared to pure Zn/SSZ-13 catalyst. The Zn^{δ+} and Cr⁶⁺ content in the CP-synthesized Zn₃Cr₁/SSZ-13 catalyst was similar to those of pure Zn/SSZ-13 and Cr—SSZ-13 catalysts, respectively.

XANES spectra and extended X-ray absorption fine structure (EXAFS) spectra were used to study coordination structures of Zn and Cr sites. As shown in FIGS. 1F and 1G, the electron transition from Zn 1s to Zn 4p unoccupied orbitals (Feature A) is revealed in Zn K-edge XANES spectra; and Cr K-edge XANES spectra exhibits the peak of electron transition from Cr 1s to Cr 3d-O 2p unoccupied orbitals (Feature B). As the Zn/Cr ratio increases from 1/1 to 4/1 in ZnₓCr_{y}/SSZ-13 catalysts, the intensity of feature A decreases, suggesting electron occupation in Zn 4p unoccupied orbitals, while the intensity of feature B increases, which suggests electrons transfer out of Cr 3d-O 2p orbitals near the conduction band minimum. It is hypothesized that a link between these observations, that electronic charge transfers from Cr 3d-O 2p character in the conduction band to Zn 4p, due to a strong Zn—Cr interaction. Thus, the charge transfer decreases the oxidative state of Zn²⁺ to Zn^{δ+} (0<δ<2), which is consistent with XPS and Auger results (FIG. 1E).

In FIG. 1H, the scattering peaks at 1.50 Å were assigned to Zn—O coordination in the first shell. The pure Zn/SSZ-13 and ZnₓCr_{y}/SSZ-13 catalysts exhibited a significantly lower intensity for this Zn—O scattering peak than the ZnO reference, suggesting a higher degree of crystal disorder. The scattering peaks at ˜2.85 Å were assigned to Zn—O—Zn coordination in the second shell. Compared to Zn/SSZ-13 and the ZnO reference, Zn₁Cr₁/SSZ-13 and Zn₃Cr₁/SSZ-13 exhibited no peak for Zn—O—Zn coordination. Instead, a new peak at ˜3.08 Å should be attributed to the Zn—O—Cr bond. Differently, the Zn₄Cr₁/SSZ-13 catalyst has both Zn—O—Cr and Zn—O—Zn bonds.

Cr K-edge Fourier transformed EXAFS (FT-EXAFS) spectra of Cr-containing catalysts showed Cr—O scattering peaks located at 1.51 Å, which is similar to standard peak of Cr—O scattering 1.50 Å in Cr^{III}₂O₃; but as Zn/Cr ratio increased to 3/1 and 4/1, a visible shoulder peak corresponding to standard peak of Cr—O scattering at 1.20 Å in K₂Cr^{VI}₂O₇ appeared, indicating that Zn₃Cr₁/SSZ-13 and Zn₄Cr₁/SSZ-13 catalysts have two types of Cr—O coordination.

ZnₓCr_{y}/SSZ-13 catalyst (x/y=1, 3, 4) catalysts showed Zn—O—Cr coordination with a bond distance ˜3.4 Å in phase-corrected space, which corresponds to the emergent peak at ˜3.08 Å in FIG. 1H. These results provide evidence for the formation of a Zn^{δ+}—O—Cr⁶⁺ structure in ZnₓCr_{y}/SSZ-13. Note that ZnₓCr_{y}/SSZ-13 catalysts also exhibited lower coordination numbers (CN) for Zn—O and Zn—O—Cr compared to the pure Zn/SSZ-13 catalyst (FIG. 1I), which results from Cr colocalization. Especially for the Zn₃Cr₁/SSZ-13 and Zn₄Cr₁/SSZ-13 catalysts, the CNs of Zn—O and Zn—O—Cr are 3.94 and 0.86, and 3.77 and 1.07, respectively. Zn₃Cr₁/SSZ-13 has the highest CN for the Cr—O bond in hexavalent (Cr⁶⁺) states. These results confirm the formation of hetero binuclear Zn^{δ+}—O—Cr⁶⁺ sites, and Zn₃Cr₁/SSZ-13 exhibits the highest population of the Zn^{δ+}—O—Cr⁶⁺ site.

### Example—Catalytic Performance of Zn—O—Cr ABC for ICEC

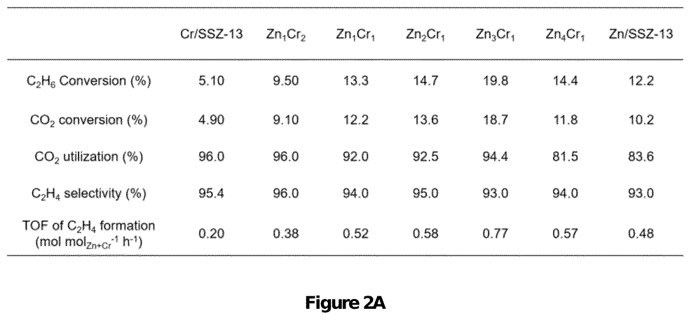

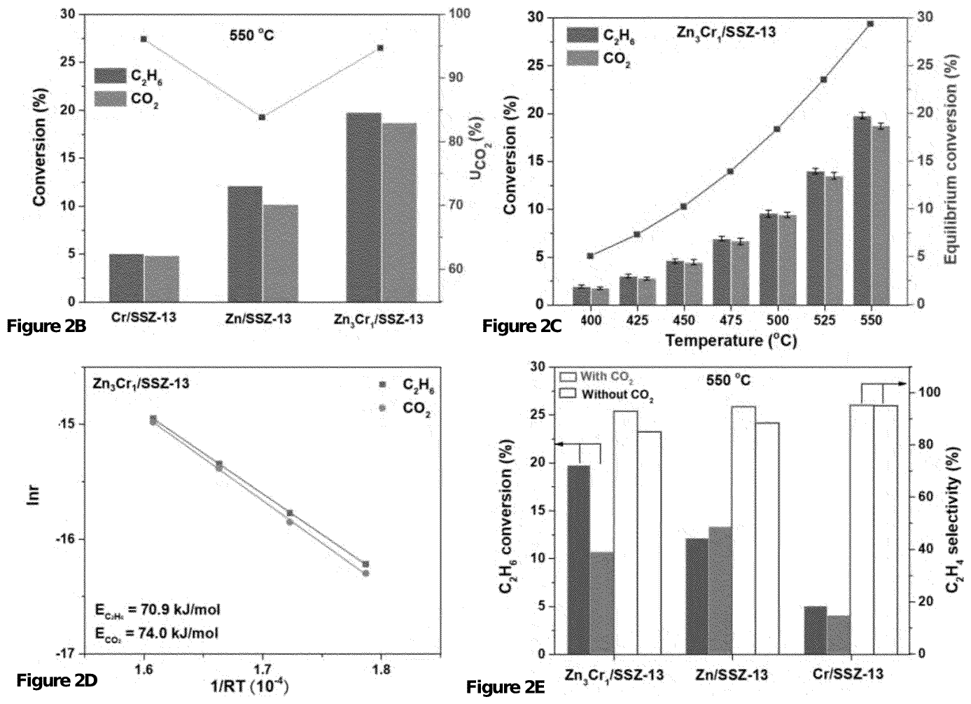

FIGS. 2A and 2B show the ICEC catalytic performance of Zn—O—Cr ABCs under the reaction conditions of weight hourly space velocity (WHSV) of 7500 mL g_{cat}⁻¹ h⁻¹ and a CO₂/ethane ratio of 1 at 550° C. The Zn₃Cr₁/SSZ-13 ABC catalyst displayed the best performance among all of the tested catalysts, with the highest C₂H₆ conversion (19.8%), CO₂ conversion (18.7%), and turnover frequency (TOF) of C₂H₄ formation (0.77 mol mol_{Zn+Cr}⁻¹ h⁻¹), as well as excellent C₂H₄ selectivity (93.0%) and U_{CO}_{₂} (94.4%).

Previous studies have mainly focused on achieving high ethane conversion and ethylene selectivity in the co-conversion of ethane and CO₂ by co-feeding excess CO₂ at CO₂/ethane ratios of 2 to 6. In the ICEC process the C₂H₄ selectivity and U_{CO}_{₂} are two important factors for evaluating the success of the catalyst development. Utilization of converted CO₂ (U_{CO}_{₂}) is defined as the ratio of CO₂ conversion to ethane conversion on an iso-stoichiometric basis.

Compared with previous studies, the Zn₃Cr₁/SSZ-13 catalyst displays the highest space-time yield (STY) of C₂H₄ formation (0.086 kg h⁻¹ kg_{cat}⁻¹) and the highest U_{CO}_{₂} at 550° C., which clearly shows the superiority of Zn—O—Cr ABCs. Further, FIG. 2C shows C₂H₄ selectivity can achieve 100% and U_{CO}_{₂} is 99.0% under the reaction temperature of 500° C., with ethane conversion of 9.6% and CO₂ conversion of 9.5%. These results further demonstrate the advantage of Zn—O—Cr ABCs suitable for ICEC. In ICEC, the reaction system includes the reactants' (ethane and CO₂) adsorption, activation, reaction, and products' (ethylene, CO, and H₂O) formation, and desorption. The apparent activation energies (Ea) for ethane conversion and CO₂ conversion are key to evaluating the rate matching of both reactions. FIG. 2D shows similar Ea (results were calculated based on FIG. 2C) for ethane dehydrogenation (70.9 KJ/mol) and CO₂ hydrogenation (74.0 KJ/mol). This further demonstrates the feasibility and success of the Zn—O—Cr ABCs concept for desired ICEC catalyst development.

FIG. 2A shows that there is a correlation between the proportion of Zn^{δ+}—O—Cr⁶⁺ sites and ICEC performance. Pure Cr/SSZ-13 catalyst had slightly higher ethylene selectivity (95.4%) and higher U_{CO}_{₂} (96.0%) than Zn₃Cr₁/SSZ-13 catalyst, but had the lowest C₂H₆ conversion (5.1%), CO₂ conversion (4.9%), and TOF of C₂H₄ formation (0.20 mol mol_{Zn+Cr}⁻¹ h⁻¹). Increasing Zn content in Zn—O—Cr ABCs, with Zn/Cr ratios from 1/2 to 3/1, had little effect on ethylene selectivity and U_{CO}_{₂}. Instead, increased Zn content promoted higher conversion of C₂H₆ and CO₂, and led to increased TOF of ethylene formation. Further increasing the Zn content to Zn/Cr ratio of 4/1 resulted in lower ethane (14.4%) and CO₂ (11.7%) conversion, resulting in a much lower U_{CO}_{₂} (81.5%). Pure Zn/SSZ-13 exhibited further decreased C₂H₆ (12.1%) and CO₂ (10.2%) conversion compared with Zn₄Cr₁/SSZ-13. Therefore, it is hypothesized that the Zn^{δ+} in the binuclear site of Zn^{δ+}—O—Cr⁶⁺ is the primary active site for C₂H₆ dehydrogenation.

Interestingly, the Zn^{δ+} proportion also showed a correlation with CO₂ conversion, which indicates that the Zn^{δ+} sites are also involved in CO₂ adsorption, activation, or reaction. In previous studies, binuclear Zn—O—Zn catalysts have been reported to have a high activity in ethane or propane dehydrogenation, but lack the capacity of efficient CO₂ activation, leading to insufficient CO₂ utilization. In this study, the Zn₃Cr₁/SSZ-13 ABC with the highest amount of Zn^{δ+}—O—Cr⁶⁺ sites displayed ˜1.5 and ˜4-fold higher ethane dehydrogenation and CO₂ conversion performance than pure Zn and Cr catalysts, respectively (FIG. 2B), which indicates the Cr⁶⁺ site of Zn^{δ+}—O—Cr⁶⁺ is also involved in the activation and reaction of C₂H₆ and CO₂. From the above analyses, it is concluded that the unique performance of Zn—O—Cr ABCs for ICEC is due to the atomic synergies within the Zn^{δ+}—O—Cr⁶⁺ site.

To study the atomic synergies between Zn^{δ+}—O—Cr⁶⁺ site in ICEC, C₂H₆ dehydrogenation performance of Zn₃Cr₁/SSZ-13 with pure Zn/SSZ-13 and Cr/SSZ-13 catalysts in the presence and absence of CO₂ were compared. As shown in FIG. 2E, the CO₂ co-feeding significantly improved C₂H₆ conversion (19.8% vs. 10.7%) and C₂H₄ selectivity (93.0% vs. 85.0%) for Zn₃Cr₁/SSZ-13, with a high U_{CO}_{₂} up to 94.4%. In contrast, for Zn/SSZ-13, CO₂ addition failed to enhance its C₂H₆ conversion (12.1% vs. 13.3%) but resulted in an increase in C₂H₄ selectivity (94.6% vs. 86.0%). The higher C₂H₄ selectivity is due to competitive adsorption of CO₂ over C₂H₆, preventing the ethane cracking reaction, which has been reported previously. Compared with Zn/SSZ-13, the improved C₂H₆ conversion of the Zn₃Cr₁/SSZ-13 catalyst is due to the generation of Zn^{δ+} in Zn^{δ+}—O—Cr⁶⁺. For the Cr/SSZ-13 catalyst, CO₂ introduction resulted in a higher C₂H₆ conversion (5.1% vs 4.0%) with a high U_{CO}_{₂} of 96%, but it did not change the C₂H₄ selectivity (95.4% vs 95.1%). Cr-based catalysts have been reported to catalyze CO₂ and ethane conversion through the redox (or MvK) mechanism. CO₂ introduction could favor lattice oxygen replenishment to regenerate highly reactive Cr⁶⁺ species and shift the reaction equilibrium of C₂H₆ dehydrogenation, thus leading to a higher activity. These results indicate that a reaction synergy between ethane and CO₂ conversions could occur at the atomically synergistic Zn^{δ+}—O—Cr⁶⁺ site.

To further validate the Zn—O—Cr ABCs, the performance of Zn₃Cr₁/SSZ-13 prepared by the dry-deposition method with the catalyst synthesized by the traditional co-precipitation (CP) method were compared. The dry-deposition synthesized Zn₃Cr₁/SSZ-13 catalyst exhibited >4-fold higher ethane conversion (19.8% vs 4.5%) and higher U_{CO}_{₂} (94.4% vs 83.2%). The CP-synthesized Zn₃Cr₁/SSZ-13 catalyst contained large Zn/Cr oxide particles and separate ZnCr₂O₄ and ZnO phases, resulting in loss of the atomic synergies of the Zn^{δ+}—O—Cr⁶⁺ site, which is thought to correlate with its poor performance. The stability and regeneration ability of the Zn₃Cr₁/SSZ-13 ABC catalyst was also studied. The ethylene selectivity and U_{CO}_{₂} remained nearly 100% during a total of 150 h in 3 cycles. The decayed conversion of ethane and CO₂ can be totally regenerated by oxidative treatment in air at 500° C. This excellent durability and regeneration ability demonstrates the structural stability of Zn^{δ+}—O—Cr⁶⁺ site.

### Example—Electronic Structure of Binuclear Zn^{δ+}—O—Cr⁶⁺ Sites During the Reaction

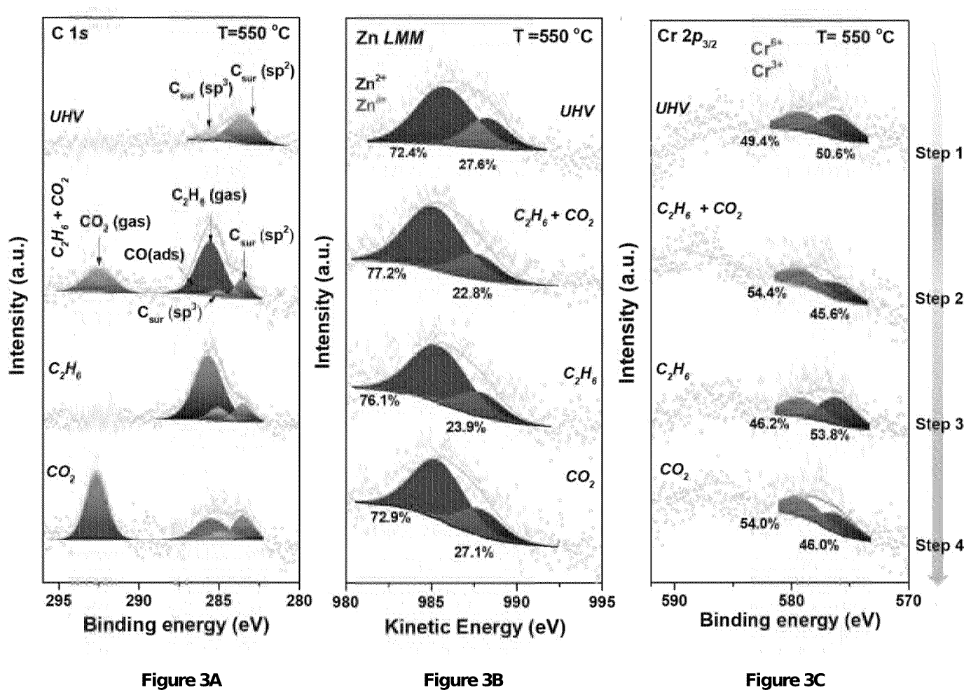

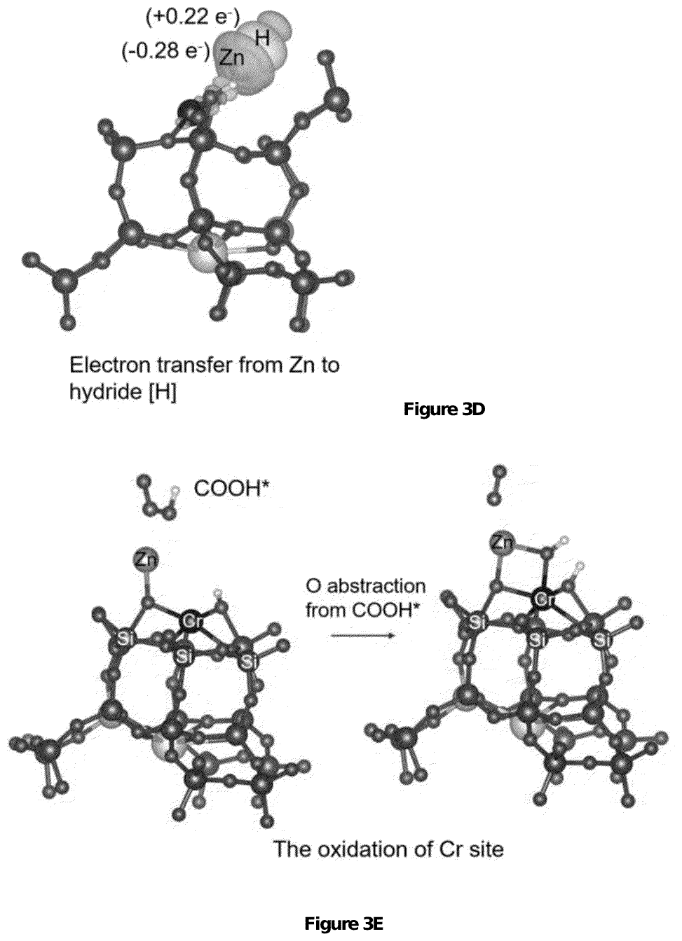

In situ ambient pressure X-ray photoelectron spectroscopy (APXPS) was used to examine the electronic structure of binuclear Zn^{δ+}—O—Cr⁶⁺ sites and study the atomic synergies between Zn^{δ+} and Cr⁶⁺ sites in ICEC. FIGS. 3A-3C show ambient pressure spectra indicating C 1s binding energies, Zn LMM Auger kinetic energies, and Cr 2p_{3/2} binding energies.

Step 1: Zn₃Cr₁/SSZ-13 was first tested under 550° C. in an ultra-high vacuum (UHV). The temperature of 550° C. was used to match the reaction temperature used in the experiments used to generate FIGS. 2A-2E. 2. In Auger spectra of Zn LMM (FIG. 3B), both Zn²⁺ and Zn^{δ+} species were detected with a percentage distribution of 72.4% and 27.6%, respectively. As shown in FIG. 3C, Cr³⁺ and Cr⁶⁺ were detected with a proportion of 50.6% and 49.4%, respectively.

Step 2: The Zn₃Cr₁/SSZ-13 was subjected to simulated reaction conditions of ICEC by co-feeding 50 mTorr C₂H₆ and 50 mTorr CO₂ under 550° C. The signals of gaseous C₂H₆ and CO₂ were detected as shown in FIG. 3A. Notably, adsorbed CO species (CO_{ads}) at 285.5 eV were observed, which indicates the ICEC reaction occurred. Under the reaction conditions, the proportion of Zn^{δ+} decreased (from 27.6% to 22.8%) and Cr⁶⁺ ratio increased (from 49.4% to 54.4%). DFT calculations (FIG. 3D) indicate that the oxidation from Zn^{δ+} to Zn²⁺ is due to the formation of Zn—H^{δ-} hydride during the ethane dehydrogenation, with more negative charge accumulated on H^{δ-}. The oxidation of Cr³⁺ to Cr⁶⁺ may result from lattice oxygen replenishment via CO₂ dissociation. These results indicate both Zn^{δ+} and Cr⁶⁺ were involved in the ICEC reaction.

Steps 3 and 4: Steps 3 and 4 were designed to help to understand the role of individual Zn^{δ+} or Cr⁶⁺ in ICEC. When feeding 100 mTorr C₂H₆ (without CO₂) in step 3 (FIGS. 3A and 3B), the signals of CO₂ and CO_{ads} species disappeared, which indicates that only ethane dehydrogenation could occur. The proportions of Zn²⁺ and Zn^{δ+} remained similar to the case of co-feeding C₂H₆ and CO₂ in step 2, indicating the formation of Zn—H^{δ-} hydride during the ethane dehydrogenation. Notice that the proportions of Zn^{δ+} and Zn²⁺ are comparable between step 1 (UHV) and step 4 (feeding 100 mTorr CO₂ without ethane), ruling out the likelihood of oxidation of Zn^{δ+} to Zn²⁺ by CO₂ or its derived intermediates.

In the case of Cr 2p3/2 XPS spectra (FIG. 3C), the oxidation of Cr³⁺ to Cr⁶⁺ only occurred when CO₂ was fed (steps 2 and 4). Previous studies have demonstrated the oxidation of Cr³⁺ to Cr⁶⁺ in Cr-based catalysts suffers from sluggish O abstraction from CO₂. In the cases shown here, the facile reoxidation of Cr³⁺ to Cr⁶⁺ may be due to the possibility that nearby Zn^{δ+} facilitate the CO₂ activation to enable easier O abstraction. The CO_{ads} species at 285.5 eV were detected in the presence/co-presence of CO₂ in the C 1s spectra (FIG. 3A, steps 2 and 4), which further demonstrates facile CO₂ dissociation over the Zn^{δ+}—O—Cr⁶⁺ site. The DFT calculation (FIG. 3E) indicates that the intermediate of CO₂ activation is carboxyl (COOH*). The decomposition of COOH* requires the participation of the Cr site through the formation of a new Cr—O bond (d(Cr—O)=2.08 Å), which will maintain its high oxidation state. In summary, the APXPS results show that binuclear Zn^{δ+}—O—Cr⁶⁺ sites serve as atomically synergistic sites for ICEC.

### Example—Atomically Synergistic Mechanism

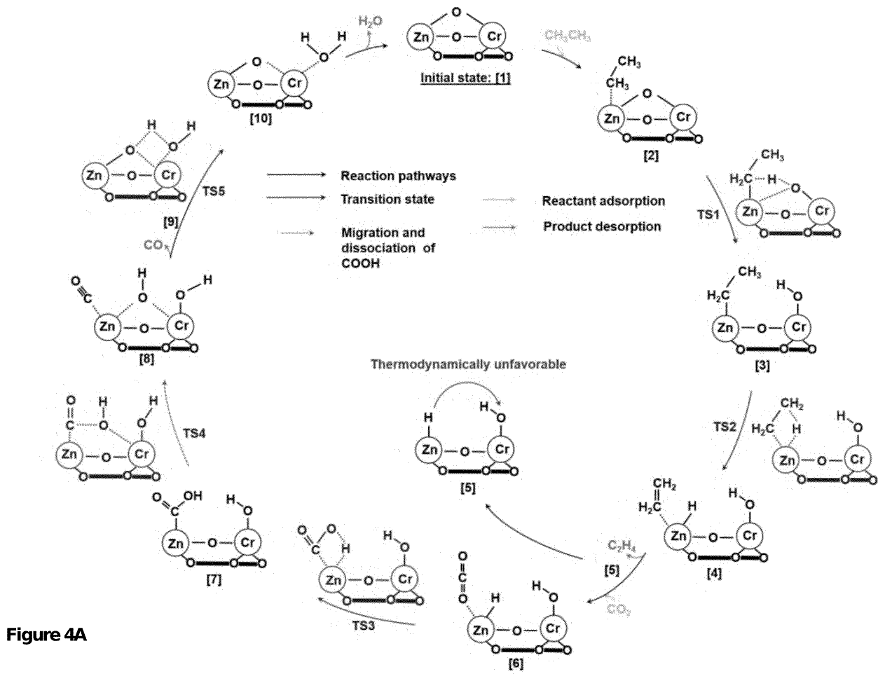

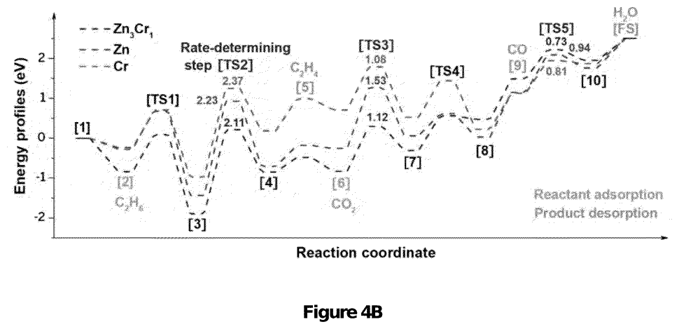

Atomically synergistic mechanisms for ICEC on binuclear Zn—O—Cr sites were developed. FIG. 4A shows that the catalytic cycle is initiated by C₂H₆ adsorption on Zn ([1]→[2]), then heterolytic cleavage of the first C—H bond (0.93 eV) by breaking a Zn—O—Cr bond to form Zn—CH₂—CH₃ and Cr—OH ([2]→[3]), followed by a homolytic scission of the β-C—H bond (2.11 eV) ([3]→[4]), and finally, C₂H₄ desorption and Zn-H^{δ-} hydride formation ([4]→[5]). C₂H₆ activation at the Cr of Zn—O—Cr sites has higher energy barriers for the first C—H cleavage (1.78 eV vs 0.93 eV) and β-C—H bond dissociation (>3.0 eV vs 2.11 eV), leading to a kinetically unfavorable pathway. In the second stage (FIG. 4A), CO₂ prefers to adsorb at the Zn—H^{δ-} site (−0.35 eV) than the Cr site (−0.05 eV) through acid-base interaction ([5]→[6]); CO₂ adsorption energy (−0.35 eV) at Zn—H^{δ-} site is lower than ethane adsorption (−0.17 eV), which will help prevent the C₂H₆ cracking reaction. Then Zn—H^{δ-} enables CO₂ hydrogenation ([6]→[7]) to carboxyl (COOH*), followed by CO—OH cleavage and migration of —OH to be shared with Cr ([7]→[8]). Finally, after CO desorption from the Zn site and the formation and desorption of H₂O from the Cr site to replenish lattice oxygen ([8]→[10]), the ICEC catalytic cycle is closed.

Calculated energy profiles of ICEC over Zn₃Cr₁/SSZ-13, Zn/SSZ-13, and Cr/SSZ-13 are shown in FIG. 4B. The rate-determining step of ICEC is the homolytic cleavage of the β-C—H bond in ethane activation [TS2]. Zn₃Cr₁/SSZ-13 exhibited a lower activation barrier (2.11 eV) than Zn/SSZ-13 (2.37 eV) and Cr/SSZ-13 (2.23 eV). Moreover, Zn₃Cr₁/SSZ-13 also had a lower energy barrier (1.12 eV, 0.73 eV) for CO₂ activation [TS3] and H₂O formation [TS5] than Zn/SSZ-13 (1.53 eV, 0.94 eV). This explains the higher CO₂ conversion and utilization of Zn₃Cr₁/SSZ-13 catalyst than Zn/SSZ-13 catalyst (FIG. 2B). Zn₃Cr₁/SSZ-13 exhibited the most favorable energetics of reactants adsorption and products desorption which also helps accelerate catalytic reactions. Therefore, the kinetically and thermodynamically favorable elementary steps in the ICEC catalytic cycle results in the high catalytic performance of Zn₃Cr₁/SSZ-13. Its performance in ICEC is due to the atomic synergies between the acidic Zn site and the redox Cr site of ABC.

### CONCLUSION

The advantages of an atomically-synergistic binuclear-site catalyst (ABC) synthesized by colocalizing Zn and Cr sites on a zeolite SSZ-13 support (ZnCr/SSZ-13) for the iso-stoichiometric co-conversion of ethane and CO₂ (ICEC) reaction has been demonstrated. This ZnCr ABC catalyst exhibited exceptional catalytic performance, with 100% ethylene selectivity and 99.0% U_{CO}_{₂} under optimized conditions. The combined results of XAS, AP-XPS, and DFT studies show that the electronic properties and catalytic activity of ZnCr/SSZ-13 can be assigned to unique atomic synergies between neighboring Zn and Cr atoms resulting from colocalization. The redox Cr site facilitates the formation of Zn^{δ+}, which is the active site for easier adsorption and activation of both ethane and CO₂. Further, the Cr site accelerates CO₂ dissociation, due to its redox properties, and facilitates the formation and desorption of H₂O. This combination results in rate matching between ethane dehydrogenation and CO₂ hydrogenation. This highlights the importance of atomic synergy, providing guidance for developing novel catalysts with potential economic and ecological benefits in CO₂ conversion and olefin production.

In the foregoing specification, the invention has been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

What is claimed is:

1. A method comprising:
mixing a Zn precursor, a metal precursor, and a zeolite to form a mixture, a metal of the metal precursor being a metal from a group Cr, Mo, and W;
grinding the mixture; and
after grinding the mixture, heat treating the mixture to form a ZnxMy/zeolite catalyst, wherein 1/2≤x/y≤4, wherein Zn is an oxide, wherein M is an oxide, and wherein Zn and M form at least some ZnMOₓ oxides.

2. The method of claim 1, wherein the zeolite is a zeolite from a group HSSZ-13 zeolite, NaSSZ-13 zeolite, HZSM-5 zeolite, NaZSM-5 zeolite, H-ZSM-11 zeolite, H-ZSM-22 zeolite, H-ZSM-23 zeolite, ZSM-35 zeolite, Beta zeolite, SAPO-11 zeolite, and SAPO-34 zeolite.

3. The method of claim 1, wherein the Zn precursor is a Zn precursor from a group zinc (II) acetate, zinc (II) nitrate, and zinc (II) chloride, wherein the Cr precursor is a Cr precursor from a group chromium (III) acetate hydroxide, chromium (III) nitrate, and chromium (III) chloride, wherein the Mo precursor comprises ammonium molybdate tetrahydrate, and wherein the W precursor comprises ammonium metatungstate hydrate.

4. The method of claim 1, wherein grinding the mixture is performed with a ball mill.

5. The method of claim 1, wherein the Zn precursor and the metal precursor each have a particle size of less than about 0.5 nanometers after the grinding.

6. The method of claim 1, wherein the zeolite has a particle size of about 1 micron to 2 microns after the grinding.

7. The method of claim 1, wherein the heat treating comprises heating the mixture to about 300° C. to 800° C. for about 1.5 hours to 4.5 hours.

8. The method of claim 1, wherein the ZnxMy/zeolite catalyst is a Zn3M1/zeolite catalyst.

9. The method of claim 1, wherein the ZnxMy/zeolite catalyst is a Zn3Cr1/SSZ-13 catalyst.

10. The method of claim 1, further comprising:
prior to the mixing, dehydrating the zeolite.

11. The method of claim 10, wherein the dehydrating comprises heating the zeolite to about 60° C. to 180° C. for about 1.5 hours to 4.5 hours while under a vacuum.

12. The method of claim 1, further comprising:
after the heat treating, neutralizing the surface acidity of the zeolite.

13. The method of claim 12, wherein neutralizing the surface acidity of the zeolite comprises Na⁺-neutralizing the zeolite comprising dispersing the mixture in deionized water to form a suspension, adding a sodium bicarbonate solution to the suspension so that the suspension has a pH of about 7, and removing the mixture from the suspension.

14. A ZnxMy/zeolite catalyst, with 1/2≤x/y≤4, M being a metal from a group Cr, Mo, and W, Zn being an oxide, M being an oxide, and at least some ZnMOₓ oxides.

15. The ZnxMy oxide/zeolite catalyst of claim 14, wherein the ZnxMy/zeolite catalyst comprises a zeolite with Zn oxide nanoparticles, M oxide nanoparticles, and ZnMOₓ oxide nanoparticles disposed on the zeolite.

16. The ZnxMy oxide/zeolite catalyst of claim 14, wherein the Zn oxide nanoparticles, M oxide nanoparticles, and ZnMOₓ oxide nanoparticles have particle sizes of about 0.5 nanometers to 1 nanometer.

17. The ZnxMy/zeolite catalyst of claim 14, wherein the zeolite is a zeolite from a group HSSZ-13 zeolite, NaSSZ-13 zeolite, HZSM-5 zeolite, NaZSM-5 zeolite, H-ZSM-11 zeolite, H-ZSM-22 zeolite, H-ZSM-23 zeolite, ZSM-35 zeolite, Beta zeolite, SAPO-11 zeolite, and SAPO-34 zeolite.

18. The ZnxMy/zeolite catalyst of claim 14, the ZnxMy/zeolite catalyst is a Zn3M1/zeolite catalyst.

19. The ZnxMy/zeolite catalyst of claim 14, wherein the ZnxMy/zeolite catalyst is Zn3Cr1/SSZ-13.

20. A method comprising:
providing a ZnxMy/zeolite catalyst, with 1/2≤x/y≤4, M being a metal from a group Cr, Mo, and W, Zn being an oxide, M being an oxide, and at least some ZnMOₓ oxides;
heating the ZnxMy/zeolite catalyst to about 400° C. to 650° C.; and
flowing carbon dioxide and ethane through the ZnxMy/zeolite catalyst and generating carbon monoxide and ethylene.
